(19) United States
(12) Patent Application Publication
(10) Pub. No.: US 2025/0041190 A1
(43) Pub. Date: Feb. 6, 2025

(54) Composition and application, in particular cosmetic

(71) Applicant: MICROPHYT, BAILLARGUES (FR)

(72) Inventors: Rémi PRADELLES, MONTPELLIER (FR); Cyrielle HOUDIN, MONTPELLIER (FR)

(21) Appl. No.: 18/718,170

(22) PCT Filed: Dec. 13, 2022
(86) PCT No.: PCT/FR2022/052323

§ 371 Date: Jun. 10, 2024

(30) Foreign Application Priority Data
Dec. 14, 2021 (FR) 21/13500

(51) Int. Cl. A61K 8/49; A61K 8/36; A61K 8/365; A61K 8/63; A61K 8/92; A61K 8/9706; A61Q 19/00

(52) U.S. Cl. CPC A61K 8/4973; A61K 8/361; A61K 8/365; A61K 8/63; A61K 8/922; A61K 8/9706; A61Q 19/00; A61Q 19/005

(57) ABSTRACT

The invention relates to a composition comprising from 0.1 to 8.0 mg/g of composition of at least one xanthophyll, from 1.0 to 45.0 mg/g of composition of at least one omega-3 fatty acid, from 0.02 to 0.8 mg/g of composition of at least one sterol, from 0.05 to 1.5 μg/g of composition of at least one phycoprostane and from 700 to 990 mg/g of at least one vegetable oil. The composition is used to provide a soothing effect on the skin and/or to maintain homeostasis.

The invention relates to a composition and notably to its cosmetic application, to obtain a soothing effect on the skin and/or maintain homeostasis.

The skin is a complex organ consisting of three layers of tissue: the epidermis, the most superficial layer, consisting of epithelial tissue comprising keratinocytes and melanocytes: the dermis, the connective tissue which supports the epidermis and comprising, inter alia, fibroblasts; and the hypodermis, the adipose tissue located beneath the dermis.

While the skin constitutes the body's protective barrier against the outside world, it is also a major sensory organ. Specifically, the epidermis, dermis and hypodermis are extensively innervated, and contacts between the nerve fibers constituting cutaneous nerves and skin cells have been observed. Cutaneous nerve fibers are known to release neuromediators such as substance P, somatostatin, CGPR (Calcitonin-Gene Related Peptide), neuropeptide Y and even endorphins. Skin cells (epidermal and dermal) also produce neuromediators, and moreover express a certain number of receptors for these neuromediators.

The CGRP peptide is a pain neuromediator, like substance P. It is a 37-amino-acid peptide in two forms, alpha and beta, released mainly by non-myelinated C-fibers in the epidermis, known as free nerve endings.

The MOR or μ-opioid receptor is a neuronal endomorphic receptor which immunohistochemical studies have shown to be present in skin nerve fibers. It is thus involved in modulating the pain response in the skin, and increasing its expression makes it possible to obtain an immediate sensation of well-being by relieving pain.

The cutaneous system is in fact in close interaction with the nervous and immune systems. This is known as the neuro-immunocutaneous system (NICS). It is this system that allows homeostasis to be maintained. This is also the system that explains the cutaneous sensitivity felt by certain people, without there necessarily being any observable clinical sign. The term “sensitive skin” is frequently used. People most often describe sensations of discomfort, with one or more characteristic symptoms such as itching, tingling, tightness, warmth or pain to the touch. These manifestations originate in a neurosensory component and are due to the release of neurotransmitters by nerve fibers in the epidermis and dermis.

While ingredients already exist on the cosmetics market to try to improve this state of discomfort and pain in “sensitive” skin, there is an ongoing need for alternative, effective ingredients. The present invention proposes to meet this need by providing a composition of totally natural origin.

Surprisingly, the Applicant has discovered that a composition which comprises from 0.1 to 8.0 mg/g of at least one xanthophyll, from 1.0 to 45.0 mg/g of at least one omega-3 type fatty acid, from 0.02 to 0.8 mg/g of at least one sterol, from 0.05 to 1.5 μg/g of at least one phycoprostane and 700 to 990 mg/g of at least one plant oil have effects on the expression of certain proteins of interest in the neuro-immunocutaneous system, with the unexpected effect of soothing the skin, in particular sensitive skin. The composition also has effects on increasing the length of neuronal extensions, allowing homeostasis to be maintained.

One of the advantages of the composition according to the invention is that it can be produced from a totally natural, non-genetically modified microalgal extract, with no additive other than a plant oil. It is non-irritant to the skin and does not induce any allergic reaction.

The composition according to the invention also allows several components of the neuro-immunocutaneous system to be targeted, making it particularly effective in reducing sensations of cutaneous discomfort and pain.

Patent application WO2013032333 describes a composition based on omega-3 type fatty acids, in particular eicosapentaenoic acid (EPA) and docosahexaenoic acid (DHA), asthaxanthin and glycerophospholipids, useful for the prevention or treatment of cognitive disorders when administered orally. The ingredients are present in the composition in the form of microalgal extracts. Said patent application does not disclose any cosmetic use for such a composition, nor even a composition in different proportions of the molecules described.

Patent application JP2003063942 describes a skin composition that is useful for the treatment of sensitive skin. Said composition comprises an anti-inflammatory agent and a skin barrier function normalizing agent, allowing improved skin moisturization. Omega-3 type fatty acids are described therein but as barrier function normalizing agents. Thus, said patent application does not describe the use of an omega-3 type fatty acid that is effective in reducing cutaneous sensitivity or in providing a skin-soothing effect. Microalgal extracts are also described for their beneficial effect on the skin. Thus, patent application EP2168570 relates to the use of an extract of Tisochrysis lutea (Isochrysis sp. var. Tahiti) that is capable of modifying or influencing hair growth and/or skin and hair pigmentation.

A dermocosmetic formula described in patent application WO2015084136 may also comprise an extract of a microalgal extract of the Isochrysis genus. Said formula has several activities, including anti-inflammatory activity, antibacterial activity and antioxidant activity.

Patent application WO2020178213 also describes a nontherapeutic method for treating sensitive skin, notably to reduce redness, comprising the application of a cosmetic composition comprising a hydrophilic extract of Phaeodactylum tricornutum, in particular an aqueous extract. As this extract is hydrophilic, it cannot contain omega-3 type fatty acids or xanthophylls.

Patent application WO 02/080876 discloses the use of a Phaeodactylum tricornutum extract as a cosmetic agent promoting the proteasome activity of skin cells and for manufacturing a cosmetic composition designed to protect the skin against the harmful effects of UV exposure and to prevent and/or delay the effects of skin aging. Said patent application does not describe any soothing effect on the skin. The effect of the extract after UV exposure is an antiaging effect.

Thus, to the Applicant's knowledge, a soothing effect on the skin of a composition comprising omega-3 type fatty acids, xanthophylls, prostaglandin-type compounds and sterols has never been described. A soothing effect on the skin of an extract of microalgae of the genus Isochrysidaceae has not been described either.

A first subject of the invention thus relates to a composition comprising from 0.1 to 8.0 mg/g of composition of at least one xanthophyll, from 1.0 to 45.0 mg/g of composition of at least one omega-3 type fatty acid, from 0.02 to 0.8 mg/g of composition of at least one sterol, from 0.05 to 1.5 μg/g of composition of at least one phycoprostane and from 700 to 990 mg/g of composition of at least one plant oil. A composition of the present invention is described in the present text for cosmetic applications, but is not restricted thereto. It may thus be envisaged that it is used according to a mode of administration other than that recommended by topical application.

By way of example, it might be administered orally.

Another subject relates to the cosmetic, nontherapeutic use of the composition according to the invention to obtain a soothing effect on the skin and/or to maintain homeostasis. Yet another subject relates to a nontherapeutic cosmetic care process comprising topical application of the composition according to the invention.

A first subject of the invention thus relates to a composition comprising from 0.1 to 8.0 mg/g of composition of at least one xanthophyll, from 1.0 to 45.0 mg/g of composition of at least one omega-3 type fatty acid, from 0.02 to 0.8 mg/g of composition of at least one sterol, from 0.05 to 1.5 μg/g of composition of at least one phycoprostane and from 700 to 990 mg/g of composition of at least one plant oil.

Advantageously, the composition comprises from 0.1 to 4.0 mg/g of composition of at least one xanthophyll, from 1.0 to 20.0 mg/g of composition of at least one omega-3 type fatty acid, from 0.02 to 0.4 mg/g of composition of at least one sterol, from 0.1 to 1.2 μg/g of composition of at least one phycoprostane and from 800 to 990 mg/g of composition of at least one plant oil.

More advantageously, said composition comprises from 0.1 to 1.0 mg/g of composition of at least one xanthophyll, from 1.0 to 5.0 mg/g of composition of omega-3 type fatty acids, from 0.02 to 0.2 mg/g of composition of at least one sterol, from 0.2 to 1.0 μg/g of composition of at least one phycoprostane and from 850 to 980 mg/g of composition of at least one plant oil, and very advantageously, it comprises from 0.1 to 0.5 mg/g of composition of at least one xanthophyll, from 1.0 to 3.1 mg/g of composition of omega-3 type fatty acids, from 0.02 to 0.15 mg/g of composition of at least one sterol, from 0.35 to 0.8 μg/g of composition of at least one phycoprostane and from 950 to 980 mg/g of composition of at least one plant oil.

Omega-3 type fatty acids are a family of unsaturated fatty acids whose hydrocarbon-based chain is of the order of 4 to 36 carbon atoms, generally of the order of 14 to 36 carbon atoms, and whose double bond or first double bond, counted from the terminal methyl group of the chain, is on the third carbon-carbon bond. The unsaturation(s) may be, independently of each other, cis or trans. The most representative acids are alpha-linolenic acid (ALA), eicosapentaenoic acid (EPA) and docosahexaenoic acid (DHA), but the designation “omega-3 type fatty acids” is not restricted thereto. In addition, and in particular when the fatty acid(s) are of natural origin, they may be extracted from algae and present in the form of free molecules but also in a derived form such as an esterified form, for example in mono-, di- or tri-esterified form, or as mixtures thereof.

Preferentially for the purposes of the invention, the or at least one of the omega-3 type fatty acids is chosen from stearidonic acid (SDA), eicosapentaenoic acid (EPA), docosahexaenoic acid (DHA) and/or any of the mixtures thereof. More preferentially, the omega-3 type fatty acid is SDA.

For the purposes of the invention the term “xanthophylls” means astaxanthin, cantaxanthin, vaucheriaxanthin, lutein, zeaxanthin, diadinoxanthin, neoxanthin, loroxanthin, siphonoxanthin, diatoxanthin, violaxanthin, dinoxanthin, flavoxanthin, α-cryptoxanthin, β-cryptoxanthin, fucoxanthin and/or any one derivative thereof and/or any mixture thereof. Preferentially, this refers to fucoxanthin and/or any one of the derivatives thereof. The term “derivatives” means their esterified mono- or multi-ester form, their glycosilated form and/or any one of the mixtures thereof.

Sterols are a well-known family of lipids with a sterane core whose carbon in position 3 bears a hydroxyl group, which may be modified with an acetyl group, for example. They include natural sterols or phytosterols, and are combined in the present text under the term phycosterols. Phytosterols that may be mentioned, non-exhaustively, include 24-methylene-cholestorl, β-sitosterol, fucosterol, 5 methylene-cholesterol, isofucosterol, saringosterol, loxocholesterol acetate, crinosterol, and more particularly brassicasterol, stigmasterol and campesterol, preferentially brassicasterol.

The term “phycoprostane” means a family of naturally occurring, structurally prostaglandin-like lipids resulting from non-directly enzymatic oxidations of fatty acids naturally present in microalgal biomasses, combined in the present text under the term phycoprostanes. In particular, these compounds are chosen from phytoprostanes, isoprostanes and neuroprostanes, depending on which fatty acid has undergone the oxidation(s). Thus, these compounds may be derived from fatty acids such as α-linolenic acid (ALA), arachidonic acid (ARA), eicosapentaenoic acid (EPA), or docosahexaenoic acid (DHA). Phytoprostanes are mainly derived from ALA and may be chosen from 9-epi-9F It-PhytoP, ent-16-epi-16-Flt-PhytoP, 9-Flt-PhytoP, ent-16BIt-PhytoP, ent-9LIt-PhytoP. 16 (RS)-16-Alt-PhytoP. Isoprostanes are mainly derived from ARA and EPA and may be chosen from 15-E2t-IsoP, 15-F2t-IsoP, 15-epi-15-F2t-IsoP, 5-F2t-IsoP, 8 (RS)-8-F3t-IsoP. Neuroprostanes are mainly derived from DHA and may be chosen from 4-F3t-NeuroP, 10-F4t-NeuroP, 10-epi-10-F4t-NeuroP, 4 (RS)-4-F4t-NeuroP, 14 (RS)-14-F4t-NeuroP, 20 (R)-20-F4t-NeuroP.

Advantageously, for the purposes of the invention, the or at least one of the phycoprostanes is chosen from phytoprostanes, isoprostanes and neuroprostanes, more advantageously neuroprostanes.

The term “plant oil” means any oil extracted from a plant or algae, including microalgae, notably chosen from olive oil, rapeseed oil, linseed oil, sunflower oil, medium-chain triglyceride (MCT) oil. The term Medium Chain Triglycerides (MCT) means saturated fatty acid esters of glycerol with a hydrocarbon-based chain of 6 to 12 carbon atoms. An MCT oil may thus be chosen from coconut oil, advantageously coconut kernel oil, palm kernel oil and palm oil, but may also be obtained from other fats or oils. Advantageously. 30 for the purposes of the invention, the plant oil is coconut oil, very advantageously coconut kernel oil.

Thus, a preferred composition according to the invention comprises:
from 0.1 to 8.0 mg/g of composition, preferentially from 0.1 to 4.0 mg/g, very preferentially from 0.1 to 1.0 mg/g and very advantageously from 0.1 to 0.5 mg/g of fucoxanthin,
from 1.0 to 45.0 mg/g of composition, preferentially from 1.0 to 20.0 mg/g, very preferentially from 1.0 to 5.0 mg/g and very advantageously from 1.0 to 3.1 mg/g of at least one omega-3 type fatty acid chosen from stearidonic acid (SDA), eicosapentaenoic acid (EPA), docosahexaenoic acid (DHA) and/or any one of the mixtures thereof,
from 0.02 to 0.8 mg/g of composition, preferentially from 0.02 to 0.4 mg/g, very preferentially from 0.02 to 0.2 mg/g and very advantageously from 0.02 to 0.15 mg/g of at least one phycosterol,
from 0.05 to 1.5 μg/g of composition, preferentially from 0.1 to 1.2 μg/g, very preferentially from 0.2 to 1.0 μg/g and very advantageously from 0.35 to 0.8 μg/g of at least one phycoprostane chosen from phytoprostanes, isoprostanes and neuroprostanes,
from 700 to 990 mg/g of composition, preferentially from 800 to 990 mg/g and very preferentially from 850 to 980 mg/g of coconut oil, preferably coconut kernel oil.

Another preferred composition comprises:
from 0.1 to 8.0 mg/g of composition, preferentially from 0.1 to 4.0 mg/g, very preferentially from 0.1 to 1.0 mg/g and very advantageously from 0.1 to 0.5 mg/g of fucoxanthin,
from 1.0 to 45.0 mg/g of composition, preferentially from 1.0 to 20.0 mg/g, very preferentially from 1.0 to 5.0 mg/g and very advantageously from 1.0 to 3.1 mg/g of at least stearidonic acid (SDA),
from 0.02 to 0.8 mg/g of composition, preferentially from 0.02 to 0.4 mg/g, very preferentially from 0.02 to 0.2 mg/g and very advantageously from 0.02 to 0.15 mg/g of at least one phycosterol,
from 0.05 to 1.5 μg/g of composition, preferentially from 0.1 to 1.2 μg/g, very preferentially from 0.2 to 1.0 μg/g and very advantageously from 0.35 to 0.8 μg/g of at least one phycoprostane chosen from phytoprostanes, isoprostanes and neuroprostanes,
from 700 to 990 mg/g of composition, preferentially from 800 to 990 mg/g and very preferentially from 850 to 980 mg/g of coconut oil, preferably coconut kernel oil.

Yet another preferred composition comprises:
from 0.1 to 8.0 mg/g of composition, preferentially from 0.1 to 4.0 mg/g, very preferentially from 0.1 to 1.0 mg/g and very advantageously from 0.1 to 0.5 mg/g of fucoxanthin,
from 1.0 to 45.0 mg/g of composition, preferentially from 1.0 to 20.0 mg/g, very preferentially from 1.0 to 5.0 mg/g and very advantageously from 1.0 to 3.1 mg/g of at least stearidonic acid (SDA),
from 0.02 to 0.8 mg/g of composition, preferentially from 0.02 to 0.4 mg/g, very preferentially from 0.02 to 0.2 mg/g and very advantageously from 0.02 to 0.15 mg/g of at least brassicasterol,
from 0.05 to 1.5 μg/g of composition, preferentially from 0.1 to 1.2 μg/g, very preferentially from 0.2 to 1.0 μg/g and very advantageously from 0.35 to 0.8 μg/g of at least one phycoprostane chosen from phytoprostanes, isoprostanes and neuroprostanes,
from 700 to 990 mg/g of composition, preferentially from 800 to 990 mg/g and very preferentially from 850 to 980 mg/g of coconut oil, preferably coconut kernel oil.

The composition may also comprise at least one cosmetically acceptable excipient chosen from natural oils such as argan oil, shea oil, jojoba oil, avocado oil, sweet almond oil, preserving agents, emulsifiers, emollients, surfactants, moisturizers, thickeners, texturizing agents, conditioners, gloss agents, texture agents, film-forming agents, pigments, dyes, fragrances, antimicrobial agents, biological additives, chelating agents, biocidal agents, astringents, polymers, reducing agents, pH regulating agents, humectants and conditioning agents.

The composition according to the invention is available in any form suitable for cosmetic use, and preferentially in a form chosen from a cream, serum, gel, soap, dermatological bar, shower gel, aqueous or oily solution, oil-in-water emulsion or water-in-oil emulsion, mask, lotion, ointment or foam. Thus, the invention also relates to a cosmetic composition corresponding to any one or more of the characteristics described above for a composition of the invention, whether considered alone or in combination. Another subject of the invention relates to the cosmetic use of the composition according to the invention to obtain a soothing effect on the skin and/or to maintain homeostasis. Preferentially, for the purposes of the invention, the composition is not used to moisturize the skin.

The term “cosmetic use” means nontherapeutic, i.e. non-pharmaceutical and non-dermatological use: in this indication, it thus has no therapeutic purpose and will advantageously be applied to all or a portion of healthy skin: it is not, however, limited thereto, and may indeed be applied to all or a portion of damaged skin without conferring a therapeutic effect.

The term “healthy skin” means any area of skin qualified as non-pathological by a dermatologist, i.e. an area of skin free from injury, redness, infection, scarring, allergy, wound, disease, eczema, inflammation, acne and/or dermatitis.

The composition according to the invention is advantageously applied topically. Thus, in a cosmetic indication, a composition of the invention is in a form suitable for topical application. For the purposes of the invention the term “topical application” means the local application of the composition to the skin or its vaporization on the skin surface. The composition is a topically acceptable composition, i.e. a non-toxic composition which is non-irritant to the skin and which does not induce an allergic reaction. It may moreover comprise at least one cosmetically acceptable excipient.

The composition may be applied topically to at least one area of skin chosen from any area of the face and/or body. The term “any area of the body” means any area of the arms, legs, thighs, back, torso, neck, feet and/or hands, including the scalp.

The term “obtaining a soothing effect” means inducing an immediate effect of well-being on the skin, a calming effect, advantageously on healthy skin, very advantageously on skin described as sensitive.

The term “sensitive skin” means healthy skin presenting sensations of pain and/or burning, the characteristic signs of which are itching and/or stinging and/or tautness and/or tingling and/or discomfort, but with no visible clinical signs, i.e. no redness, eczema, dermatitis, desquamation or plaque. Thus sensitive skin for the purposes of the invention is not allergic skin.

In one embodiment of the invention, the term “providing a soothing effect” means reducing the gene and/or protein amount of the neuropeptide CGRP (Calcitonin Gene-Related Peptide), also known as CALCA (Calcitonin related polypeptide alpha), a known pain neuromediator, in the presence of the composition according to the invention. Advantageously, there is a reduction of at least 22% in the presence of the composition according to the invention, preferentially of at least 30%, even more preferentially of at least 40% in the protein amount of CGRP, compared with the CGRP protein amount measured without addition of the composition according to the invention but in a co-culture of keratinocytes and sensory neurons subjected to oxidative stress in the presence of capsaicin.

The term “sensory neurons” means herein human neurons derived from reprogrammed human fibroblast cells. These neurons express the capsaicin receptor TRPVI (transient receptor potential cation channel subfamily V member 1), TrkA and MOPr, and synthesize substance P and the neuropeptide CGRP in their cytoplasm. They depolarize in the presence of capsaicin.

More advantageously, the protein reduction of the CGRP neuropeptide is measured in the presence of a composition concentration of 0.1%. 0.05% or 0.01% by weight relative to the total weight of the sample. Very preferentially, the measurement of the CGRP protein amount is performed by ELISA assay under conditions as described in Example 2a) [Table 1].

Alternatively, there is a reduction in the CGRP protein amount measured in a co-culture of keratinocytes and sensory neurons subjected to oxidative stress in the presence of AITC (allyl isothiocyanate). Thus, advantageously, the CGRP protein reduction is at least 16% in the presence of the composition according to the invention, preferentially at least 30%, more preferentially at least 40%, compared with the CGRP protein amount measured in said co-culture subjected to oxidative stress without the composition according to the invention. More advantageously, this refers to a CGRP neuropeptide protein reduction measured in the presence of a composition concentration of 0.05% or 0.01% by weight relative to the total weight of the sample. Very preferentially, the measurement of the CGRP protein amount is performed by ELISA assay under conditions as described in Example 2b) [Table 2].

In another embodiment of the invention, the term “providing a soothing effect” means increasing the level of gene and/or protein expression of the MOR receptor (μ-opioid receptor) in the presence of the composition according to the invention, relative to the level of gene and/or protein expression of said receptor measured in the absence of the composition (Control). In a preferential embodiment, this refers to an increase, in the absence of stress, in the protein expression level of the MOR receptor in a co-culture of sensory neurons and human keratinocytes, of at least 14%, preferentially of at least 40%, more preferentially of at least 100% and very advantageously of at least 200% relative to the protein expression level of said receptor measured in the absence of the composition (Control). Advantageously, this refers to an increase in the presence of a composition concentration of 0.05% or 0.01% by weight relative to the total weight of the sample. More advantageously, the MOR receptor protein expression level is measured by immunolabeling, under the conditions described in Example 2c) [Table 3].

In an alternative embodiment of the invention, this refers to an increase in the level of gene and/or protein expression of the MOR receptor in the presence of the composition according to the invention, relative to the level of gene and/or protein expression of said receptor measured in the absence of the composition (Control), in a co-culture of human sensory neurons and keratinocytes subjected to oxidative stress in the presence of capsaicin (Positive Control). Advantageously, this refers to an increase in the MOR receptor protein expression level of at least 70%, advantageously of at least 150%, more advantageously of at least 200% relative to the protein expression level measured in the co-culture subjected to stress in the presence of capsaicin and without the composition according to the invention. More advantageously, the increase is measured in the presence of a composition concentration of 0.05% or 0.01% by weight relative to the total weight of the sample. More advantageously, the MOR receptor protein expression level is measured by immunolabeling, under the conditions described in Example 2d) [Table 4].

Alternatively also, this refers to an increase of at least 35%, even more preferentially of at least 50% and very preferentially of at least 200% in the protein expression level of the MOR receptor in the presence of the composition according to the invention, relative to the protein expression level of said receptor measured in the absence of the composition (positive control), in a co-culture of human sensory neurons and keratinocytes subjected to oxidative stress in the presence of AITC. More advantageously, the increase is measured in the presence of a composition concentration of 0.05% or 0.01% by weight relative to the total weight of the sample. More preferentially, the MOR receptor protein expression level is measured by immunolabeling, under the conditions described in Example 2d) [Table 5].

In yet another alternative embodiment of the invention, the term “providing a soothing effect” means reducing the sensitivity of the skin measured in the presence of the composition according to the invention in the context of a clinical study (see Example 2e)). Homeostasis is defined herein as maintaining the balance between the exchange and synthesis functions of the various molecular and cellular components at skin and/or neuronal level, allowing biological parameters to be kept constant when subjected to stress or stimuli induced by changes in external conditions. Thus, for the purposes of the invention, the term “maintaining homeostasis” means keeping the neuro-immunocutaneous system (NICS) in equilibrium so that the pain experienced is effectively correlated with a negative external stimulus, and not induced by an imbalance due to a runaway NICS system uncorrelated with an external stimulus. One of the components for maintaining this homeostasis is associated with the maintenance of the neural network and, as a result, of the length of the neural extensions.

Thus, in a preferential embodiment of the invention the term “maintaining homeostasis” means maintaining and/or increasing the length of neurons in the presence of the composition according to the invention. Advantageously thus, the composition is capable of maintaining said homeostasis when the length of the neuronal extensions is increased by at least 14%, preferentially by at least 20% in the presence of the composition according to the invention, compared with the length measured without the composition in the co-culture model of keratinocytes and sensory neurons subjected to oxidative stress in the presence of capsaicin. In a particularly advantageous mode, this refers to an increase measured in the presence of a concentration of the composition of 0.05% or 0.01% by weight relative to the total weight of the sample, under the conditions as described in Example 3 ([Table 8]). In a very advantageous mode, this refers to an increase in the neuronal extension of non-myelinated C fibres. More advantageously, the composition according to the invention does not increase the length of myelinated neurites. Nor does it increase the length of non-myelinated fibers in the absence of oxidative stress.

In a particularly advantageous embodiment of the invention, the composition has no anti-inflammatory activity, i.e. notably no inhibitory activity of pro-inflammatory markers. The invention moreover relates to the use of a microalgal extract chosen from any of the taxa Pinguiophyceae, Chrysophyceae, Bacillariophyceae, Mamiellophyceae, Prymnesiophyceae, Haptophyceae, Coccolithophyceae, Isochrysidaceae 1 and Phaeodactylaceae, to prepare the composition according to the invention. Precisely, said use relates to the preparation of the composition comprising from (0.1 to 8.0 mg/g of composition of at least one xanthophyll, advantageously from 0.1 to 4.0 mg/g: from 1.0 to 45.0 mg/g of composition of omega-3 type fatty acids, advantageously from 1.0 to 20.0 mg/g: from 0.02 to 0.8 mg/g of composition of at least one sterol, advantageously from 0.02 to 0.4 mg/g; and from 0.05 to 1.5 μg/g of composition of at least one phycoprostane, advantageously from 0.1 to 1.2 μg/g, to which is added from 700 to 990 mg/g of composition, advantageously from 800 to 990 mg/g, of at least one plant oil. Very advantageously, the use of a microalgal extract chosen from any one of the taxa Pinguiophyceae, Chrysophyceae, Bacillariophyceae, Mamiellophyceae.

Prymnesiophyceae, Haptophyceae, Coccolithophyceae, Isochrysidaceae and Phaeodactylaceae comprises the preparation of the composition comprising from 0.1 to 1.0 mg/g, even more advantageously from 0.1 to 0.5 mg/g of composition, of at least one xanthophyll: from 1.0 to 5.0 mg/g, even more advantageously from 0.1 to 3.1 mg/g of composition, of omega-3 type fatty acids: from 0.02 to 0.2 mg/g, even more advantageously from 0.02 to 0.15 mg/g of composition, of at least one sterol; and from 0.2 to 1.0 μg/g, even more advantageously from 0.35 to 0.8 μg/g of composition, of at least one phycoprostane, to which is added from 850 to 980 mg/g of composition, even more advantageously from 950 to 980 mg/g, of at least one plant oil.

Preferentially, the microalgal extract comes from the microalga Tisochrysis lutea or Isochrysis galbana or Phaeodactylum tricornutum, even more preferentially T. lutea.

The term “microalgal extract” means any extract of the biomass derived from organisms capable of photosynthesis obtained via a process which directly or indirectly affords the composition of the invention. These extracts have a composition, expressed as a mass percentage of the total extract, of proteins of between 0.05% and 0.20%, sterols of between 0.005% and 0.050%, and chlorophyll of between 0.1% and 0.5%.

More precisely, the lipophilic component of the extract, advantageously T. lutea extract, expressed as a mass percentage relative to the lipophilic fraction, comprises 0.438% unsaturated fatty acids. 0.25% omega-3 acids and 0.054% omega-6 acids. Moreover, said extract comprises between 0.02% and 0.04% xanthophylls as mass percentages relative to the extract. Thus, it may comprise 0.035% fucoxanthin. The extract also comprises 2.65 mg omega-3 acids/g of extract, and 230 ng phycoprostane(s)/g of extract.

The microalgae are cultivated in a controlled manner in suitable systems such as raceways, open ponds or preferentially closed systems of the photobioreactor type. The photobioreactors used may be of any existing type, such as horizontal tubular photobioreactors, vertical photobioreactors such as “green wall panel” systems, flat photobioreactors or columnar photobioreactors. Preferentially, biomass production will be performed in a closed cultivation system, by autotrophy with no impact on arable land. Biomass production is performed according to a batch, fed-batch, continuous, semi-continuous, turbidostat or chemostat type of culturing procedure. The extracts are obtained by concentrating fresh or frozen biomass, removing all or some of the water using chemical or physical processes such as centrifugation, filtration, flocculation or sedimentation, optionally combined with drying steps such as freeze-drying, vacuum drying, drum drying, atomization or any other process that reduces the water content of the biomass. In addition to these steps, cell lysis processes may be performed, such as the application of pressure, electrical flows, shear forces, the use of enzymes, or any other process enabling the destructuring of tissues, organs, cells or organelles.

The microalgal extract may be obtained via any solid-liquid extraction-type process, possibly using hypercritical fluids or subcritical fluids, and possibly involving parallel or sequential co-treatments such as microwaves, ultrasound, pressure or enzymes. The microalgal extract may thus be obtained by extraction under subcritical conditions.

Extraction may be performed in the presence of any suitable solvent chosen from acetone. hexane, ethyl acetate, methyltetrahydrofuran, heptane, methanol, a natural or branched oil, ethanol or any other solvent enabling all or some of the hydrophobic and amphiphilic compounds to be extracted. The solvent may be used pure or in admixture. The term “in admixture” means herein a mixture with another solvent or a mixture with water in respective solvent/water volume proportions of 99:1 (v/v) to 1:99 (v/v).

The solvent or solvent mixture is separated from the residual biomass after extraction via processes such as centrifugation or filtration, and may subsequently be concentrated, or the solvent removed, via techniques such as vacuum evaporation or any other technique enabling selective evaporation of the solvent under consideration. The extract thus obtained is lipophilic in nature while at the same time including amphiphilic molecules. In a preferred embodiment of the invention, the microalgal extract is an extract of Tisochrysis lutea or Isochrysis galbana or Phaeodactylum tricornutum, more preferentially T. lutea. Preferentially, the microalgal extract is obtained by extraction of the biomass, advantageously fresh biomass, in the presence of an ethanol:water mixture of 99:1 (v/v) to 1:99 (v/v), advantageously 98:2 (v/v) to 30:70 (v/v), more advantageously 95:5 (v/v). Advantageously, it is obtained by extraction from T. lutea biomass to obtain the composition according to the invention, under the conditions described in Example 1.

Another subject of the present invention also relates to a nontherapeutic cosmetic care process comprising oral administration or topical application, preferentially topical application, of the composition according to the invention, or of a microalgal extract chosen from any one of the taxa Pinguiophyceae, Chrysophyceae, Bacillariophyceae, Mamiellophyceae, Prymnesiophyceae, Haptophyceae, Coccolithophyceae, Isochrysidaceae and Phaeodactylaceae, preferentially Tisochrysis lutea or Isochrysis galbana or Phaeodactylum tricornutum, more preferentially T. lutea, advantageously to prepare the composition according to the invention, to obtain a soothing effect on the skin and/or to maintain homeostasis. Advantageously, the T. lutea extract used in the cosmetic care process is obtained by extraction of the biomass, advantageously fresh biomass, in the presence of an ethanol:water mixture of 99:1 (v/v) to 1:99 (v/v), advantageously 98:2 (v/v) to 30:70 (v/v), more advantageously 95:5 (v/v).

In one advantageous embodiment, the process comprises topical application of the composition to at least one area of skin, preferably healthy skin, chosen from any area of the face and/or body, i.e. any area of the arms, legs, thighs, back, torso, neck, feet and/or hands, including the scalp.

Examples referring to the description are presented below. These examples are illustrative and are not intended to limit the scope of the invention. The examples form an integral part of the present invention, and any new features relative to the prior art based on the description taken as a whole form an integral part of the invention. Unless expressly mentioned, the percentages are expressed on a weight/weight basis and the temperature is given in degrees Celsius.

### Example 1: Method for Preparing the Composition and an Extract of T. lutea

The strain used to prepare the composition according to the invention is CCAP 927/14, available from the Scottish Marine Institute via their Culture Collection of Algae and Protozoa (CCAP) bank. The geographical origin of this strain is France.

An extract is obtained by extraction in an ethanol:water mixture (95:5; v/v) from fresh biomass resulting from autotrophic photobioreactor culturing of the microalga Tisochrysis lutea. It is insoluble in water and highly viscous, making it impossible to handle at room temperature.

The extract and coconut kernel oil are brought to room temperature (25±1° C.) 24 hours prior to preparation. The extract is transferred to a centrifuge tube including the oil in such a manner that the final net mass of the mixture is about 5 g and the mass proportion is such that the extract consists of 25% of the total net mass of the mixture. The mixture is stirred for one minute using a mixing device known as a vortex. Stirring is repeated three times per mixture. A homogeneous mixture is obtained.

The composition obtained comprises:
Omega-3 type fatty acids (ALA, SDA, EPA, DHA): 2.65 mg/g of composition:
Fucoxanthin: 0.35 mg/g of composition:
Sterols 0.115 mg/g of composition:
Phycoprostane(s): 230 ng/g of composition; and
Coconut kernel oil: 950 mg/g of composition.

### Example 2: Soothing Effect of the Composition

### Example 2a): Protein Reduction of the Neuropeptide CGRP in a Co-Culture of Keratinocytes and Sensory Neurons Subjected to Oxidative Stress in the Presence of Capsaicin

### Protocol: Co-Culturing of Neurons and Keratinocytes:

Sensory neurons were obtained from hiPS cells (human induced pluripotent stem cells), which were themselves obtained from human fibroblasts. The cells were seeded and maintained for 6 days in a differentiation-inducing medium at 37° C. (5% CO2). The culture medium was changed every 2 days. After 9 days in culture, the culture medium was replaced with a maturation medium. Cells were maintained in culture at a temperature of 37° C. under 5% CO2. The culture medium was changed every 2-3 days. After 14 days in culture, commercial keratinocytes from a 29-year-old adult donor (Lonza) were added to the differentiated hiPS cell culture. The co-culture was maintained in a culture medium consisting of a mixture of maturation medium and keratinocyte growth medium (Promocell) at a temperature of 37° C. under 5% CO2. The culture medium was changed every 2-3 days.

Treatment of the Co-Culture with the Composition According to the Invention:

After a 17-day co-culture period, the composition was added to the medium at a final concentration by weight relative to the total weight of the medium and composition of 0.01% or 0.05% (w/w).

After a 24-hour incubation period, capsaicin (10 UM) or AITC (1 mM) or DMSO (0.2% w/w) as a control, was added to the co-culture in the presence of the composition according to the invention.

CGRP assay: After a 30-minute stress period, the culture supernatants were recovered and a CGRP assay by ELISA was performed. The results are expressed as the mean of 6 samples (n=6).

### Results:

TABLE 1

| | Protein | Standard |
|---|---|---|
| | amount of | error of |
| | CGRP (%) | mean |
| Unstressed control (DMSO 0.2%) | 100 | 0 |
| Positive control (capsaicin 10 μM) | 135 | 5 |
| Capsaicin (10 μM) and composition | 105**** | 3 |
| according to Example 1 at 0.01% (w/w) | | |
| Capsaicin (10 μM) and composition | 103**** | 2 |
| according to Example 1 at 0.05% (w/w) | | |
| Capsaicin (10 μM) and composition | 101**** | 2 |
| according to Example 1 at 0.1% (w/w) | | |

Conclusion: the composition according to the invention enabled a significant reduction in the protein amount of the CGRP neuropeptide in the co-culture model subjected to oxidative stress in the presence of capsaicin, demonstrating the positive soothing effect of the composition at the concentrations tested (**** p<<0.0001 one-way Anova test with Dunnett correction).

### Example 2b): Protein Reduction of the Neuropeptide CGRP in a Co-Culture of Keratinocytes and Sensory Neurons Subjected to Oxidative Stress in the Presence of AITC

Protocol: the protocol is that described in Example 2a).

### Results:

TABLE 2

| | Protein | Standard |
|---|---|---|
| | amount of | error of |
| | CGRP (%) | mean |
| Unstressed control (DMSO 0.2%) | 100 | 0 |
| Positive control (AITC 1 mM) | 132 | 8 |
| AITC (1 mM) and composition | 104**** | 4 |
| according to Example 1 at 0.01% (w/w) | | |
| AITC (1 mM) and composition | 97**** | 2 |
| according to Example 1 at 0.05% (w/w) | | |

Conclusion: the composition enabled a significant reduction in the protein amount of the CGRP neuropeptide in the co-culture model subjected to oxidative stress in the presence of AITC, demonstrating here also the positive soothing effect of the composition at the concentrations tested (**** p<<0.0001 one-way Anova test with Dunnett correction).

### Example 2c): Increase of MOR Receptor Protein in a Co-Culture of Unstressed Human Keratinocytes and Sensory Neurons

Protocol: the protocol for preparing the co-culture is that described in Example 2a). Immunolabeling: On conclusion of the addition or not [Table 3] of capsaicin (10 μM) [Table 4] or AITC (1 mM) [Table 5] or DMSO (0.2%) as a control, the culture supernatants were recovered and the pre-fixed cells incubated in the presence of primary antibodies raised against β-tubulin (Euromedex) and against MOR (Merck). These antibodies were revealed by fluorochrome-coupled secondary antibodies (Fisher Scientific). The nuclei were labeled with a Hoechst solution (Sigma-Aldrich), a nuclear fluorescent marker, in the same solution as the secondary antibodies.

Photographs were taken with an automated microscope (InCell 2200; GE Healthcare) at ×20 magnification. MOR marker expression density was measured and compared with the control condition, corresponding to the mean of 6 samples (n=6). Statistical analysis was performed using one-way Anova corrected with a Dunnett test.

### Results:

TABLE 3

| | Level of MOR | Standard |
|---|---|---|
| | receptor protein | error of |
| | expression (%) | mean |
| Composition-free control (DMSO 0.2%) | 100 | 7 |
| Composition according to Example 1 at | 146 | 25 |
| 0.01% (w/w) | | |
| Composition according to Example 1 at | 394**** | 33 |
| 0.05% (w/w) | | |

Conclusion: the basal level of MOR receptor protein expression was significantly increased by at least 14% in the presence of the composition according to the invention at the two concentrations tested in the absence of stress, compared with the control. Example 2d): Increase of MOR receptor protein in a co-culture of human keratinocytes and sensory neurons stressed in the presence of capsaicin [Table 4] or AICT [Table 5]

TABLE 4

| | Level of MOR | Standard |
|---|---|---|
| | receptor protein | error of |
| | expression (%) | mean |
| Composition-free control (DMSO 0.2%) | 100 | 7 |
| Positive control (capsaicin 10 μM) | 82 | 20 |
| Capsaicin 10 μM and composition | 198** | 20 |
| according to Example 1 at 0.01% (w/w) | | |
| Capsaicin 10 μM and composition | 310*** | 41 |
| according to Example 1 at 0.05% (w/w) | | |

(***p < 0.001; **p < 0.01 one-way Anova test with Dunnett correction)

TABLE 5

| | Level of MOR | Standard |
|---|---|---|
| | receptor protein | error of |
| | expression (%) | mean |
| Composition-free control (DMSO 0.2%) | 100 | 7 |
| Positive control (AITC 1 mM) | 78 | 15 |
| AITC (1 mM) and composition according | 159** | 28 |
| to Example 1 at 0.01% (w/w) | | |
| AITC (1 mM) and composition according | 303*** | 18 |
| to Example 1 at 0.05% (w/w) | | |

(***p < 0.001; **p < 0.01 one-way Anova test with Dunnett correction)

Conclusion: regardless of the oxidative stress tested (capsaicin or AITC), the composition according to the invention enabled a significant increase, of at least 70% (capsaicin) and at least 35% (AITC), in the level of MOR receptor protein expression in the co-culture studied, compared with said expression level measured in co-cultures subjected to stress. The expression levels in the presence of the composition are significantly higher than those measured in the control (unstressed) co-culture.

### Example 2e): Clinical Evaluation of the Soothing Effect of the Composition

Protocol: a clinical test was performed on a population of 42 women of Caucasian origin aged between 22 and 66 with healthy skin as defined in the present invention, but with sensitive skin. A cream comprising, by weight relative to the total weight of the cream, 0.5% of the composition as prepared in Example 1 was applied twice a day over a period of 14 days to the entire face of 21 of the 42 individuals taking part in the study. The same cream comprising 0.5% (w/w) placebo (NaOH, water, Yellow 5 dye and NATPURE COL BROWN LC816) was applied under similar conditions to the entire face of the other 21 individuals.

A measurement of vascular disorders (vascular intensity) was performed using polarized light photography with a high-resolution camera. This technique consists in obtaining high-resolution photographs of ¾ of the face under polarized light under reproducible conditions (Nikkor 60 mm). The camera lens is equipped with a filter. Light is provided via two luminous flashes. The flashes are directed onto a polarizing gel (HN32 Sarelec, France). The camera filter is positioned at 90° to the polarization of the flash filters. The polarized light emitted by the flashes is reflected by the facial skin at the moment the photograph is taken. The use of dedicated software made it possible to determine a vascular image with an intensity reflecting the effect of the application: a reduction in this intensity corresponding to a decrease in the sensitivity of the skin analyzed.

### Results:

TABLE 6
Mean vascular intensity measured in the presence
of the composition according to the invention:

| | MEAN | p (Student Test) |
|---|---|---|
| D 0 | 0.079 | |
| D 14 | 0.076 | 0.195 |
| D 14 vs D 0 | −0.003 | |
| % variation vs D 0 | −3.4 | |

TABLE 7
Mean vascular intensity measured in the presence of
the placebo composition according to the invention:

| | MEAN | p (Student Test) |
|---|---|---|
| D 0 | 0.084 | |
| D 14 | 0.087 | 0.255 |
| D 14 vs D 0 | 0.003 | |
| % variation vs D 0 | +3.5 | |

Conclusion: after 14 days of twice-daily application of the cream comprising the composition according to the invention, the mean vascular intensity of the analyzed skins decreased by at least 3.4% relative to time 0 (DO) of said application. On the contrary, the mean vascular intensity of analyzed skins to which placebo cream had been applied increased by 3.5%, reflecting an increase in skin sensitivity.

### Example 3: Effect on Increasing the Length of Neuronal Extensions

Protocol: the protocol for preparing the co-culture of sensory neurons and keratinocytes is that described in Example 2a).

On conclusion of the addition of capsaicin (10 μM) or DMSO (0.2%) as a control [Table 6], the culture superatants were recovered and the cells pre-fixed. Photographs were taken with an automated microscope (InCell 2200; GE Healthcare) at ×20 magnification.

A neuron count was performed and the length of the neuronal extensions was measured for each condition. The results were compared with the control condition (n=6). Statistical analysis was performed using a one-way Anova test followed by a Dunnett test.

### Results:

TABLE 8

| | Length of neuronal | Standard |
|---|---|---|
| | extensions/nbr of neurons | error of |
| | (%) | mean |
| Composition-free control (DMSO | 100 | 6 |
| 0.2%) | | |
| Positive control (capsaicin 10 μM) | 76 | 3 |
| Capsaicin 10 μM and composition | 101* | 8 |
| according to Example 1 at 0.01% | | |
| (w/w) | | |
| Capsaicin 10 μM and composition | 102* | 7 |
| according to Example 1 at 0.05% | | |
| (w/w) | | |

Conclusion: the composition at the concentrations tested allowed a significant increase in the length of the neuronal extensions and restored the length measured in the control not subjected to oxidative stress.
(* p<0.05; one-way Anova test with Dunnett correction)

### Example 4: Example of a Cosmetic Formulation Comprising the Composition

The components are given as weight percentages relative to the total weight of the formulation.
Caprylic/capric triglyceride 10.00
Glycerol 2.98
Myristyl myristate 2.50
Glyceryl citrate stearate 2.00
Sodium polyacrylate 0.60
Composition of the invention (Ex. 1) 0.50
1,2-Hexanediol 0.30
Caprylyl glycol 0.30
Ethylhexylglycerol 0.29
Xanthan gum 0.25
Disodium EDTA 0.10
Tocopherol 0.09
Sodium hydroxide 0.02
Helianthus annuus seed oil 0.005
Water qs 100

1. A composition comprising from 0.1 to 8.0 mg/g of composition of at least one xanthophyll, from 1.0 to 45.0 mg/g of composition of at least one omega-3 type fatty acid, from 0.02 to 0.8 mg/g of composition of at least one sterol, from 0.05 to 1.5 μg/g of composition of at least one phycoprostane and from 700 to 990 mg/g of composition of at least one plant oil.

2. The composition as claimed in claim 1, comprising from 0.1 to 4.0 mg/g of composition of at least one xanthophyll, from 1.0 to 20.0 mg/g of composition of at least one omega-3 type fatty acid, from 0.02 to 0.4 mg/g of composition of at least one sterol, from 0.1 to 1.2 μg/g of composition of at least one phycoprostane and from 800 to 990 mg/g of composition of at least one plant oil.

3. The composition as claimed in claim 1, comprising from 0.1 to 1.0 mg/g of composition of at least one xanthophyll, from 1.0 to 5.0 mg/g of composition of omega-3 type fatty acids, from 0.02 to 0.2 mg/g of composition of at least one sterol, from 0.2 to 1.0 μg/g composition of at least one phycoprostane and from 850 to 980 mg/g of composition of at least one plant oil.

4. The composition as claimed in claim 1, comprising from 0.1 to 0.5 mg/g of composition of at least one xanthophyll, from 1.0 to 3.1 mg/g of composition of omega-3 type fatty acids, from 0.02 to 0.15 mg/g of composition of at least one sterol, from 0.35 to 0.8 μg/g of composition of at least one phycoprostane and from 950 to 980 mg/g of composition of at least one plant oil.

5. The composition as claimed in claim 1, wherein at least one plant oil is chosen from olive oil, rapeseed oil, linseed oil, sunflower oil and medium-chain triglyceride (MCT) oil.

6. The composition as claimed in claim 5, wherein the medium-chain triglyceride (MCT) oil is chosen from palm oil or, coconut oil, preferentially, or coconut kernel oil.

7. The composition as claimed in claim 1, wherein the or at least one of the omega-3 type fatty acids is chosen from stearidonic acid (SDA), eicosapentaenoic acid (EPA), docosahexaenoic acid (DHA) and/or any one of the mixtures thereof, advantageously stearidonic acid.

8. The composition as claimed in claim 1, wherein the or at least one of the xanthophylls is fucoxanthin.

9. The composition as claimed in claim 1, wherein the or at least one of the sterols is chosen from phytosterols, advantageously brassicasterol.

10. The composition as claimed in claim 1, wherein the or at least one of the phycoprostanes is chosen from phytoprostanes, isoprostanes and neuroprostanes, advantageously neuroprostanes.

11. The composition as claimed in any one of claims 1 to 10, wherein it also comprises at least one cosmetically acceptable excipient.

12. The composition as claimed in claim 1, wherein it is in a form chosen from a cream, a serum, a gel, a soap, a dermatological bar, a shower gel, an aqueous or oily solution, an oil-in-water emulsion or a water-in-oil emulsion, a mask, a lotion, an ointment and a foam.

13. A microalgal extract chosen from any of the taxa Pinguiophyceae, Chrysophyceae, Bacillariophyceae, Mamiellophyceae, Prymnesiophyceae, Haptophyceae, Coccolithophyceae, Isochrysidaceae and Phaeodactylaceae, configured for preparing the composition as claimed in claim 1.

14. The microalgal extract as claimed in claim 13, wherein the microalga is Tisochrysis lutea or Isochrysis galbana, Phaeodactylum tricormitum, preferentially or Tisochrysis lutea.

15. The composition as claimed in claim 1, configured to obtain a soothing effect on the skin and/or to maintain homeostasis.

16. The composition as claimed in claim 15, further configured to obtain a soothing effect on sensitive skin.

17. A cosmetic care process comprising topical application of the composition as claimed claim 1, or of a microalgal extract chosen from any one of the taxa Pinguiophyceae, Chrysophyceae, Bacillariophyceae, Mamiellophyceae, Prymnesiophyceae, Haptophyceae, Coccolithophyceae, Isochrysidaceae and Phaeodactylaceae, Tisochrysis lutea, Isochrysis galbana or Phaeodactylum tricormitum, or Tisochrysis lutea, to obtain a soothing effect on the skin and/or to maintain homeostasis.

18. The cosmetic care process as claimed in claim 17, further comprising topical application to at least one area of skin chosen from any area of the face and/or any area of the arms, legs, thighs, back, torso, neck, feet and/or hands, including the scalp.
